(19) United States
(12) Patent Application Publication
(10) Pub. No.: US 2007/0231387 A1
(43) Pub. Date: Oct. 4, 2007

(54) Film-coated solid dosage forms

(76) Inventors: Luigi Levi, Richmond Hill (CA); Jitendra Somani, Toronto (CA); Joseph K. Lee, Toronto (CA); Arthur Paul Gerald Wright, Markham (CA)

Correspondence Address: Linda A. Vag;Warner Lambert Company LLC, 201 Tabor Road, Morris Plains, NJ, 07950, US

(21) Appl. No.: 11/398,390

(22) Filed: Apr. 4, 2006

(51) Int. Cl. A61K 9/24

(52) U.S. Cl. 424/472

(57) ABSTRACT

An orally dissolvable film coating for solid dosage forms which contains pullulan, one or more sensory cue agents, and a plasticizer. The film coating disintegrates in the mouth releasing the sensory cue agent resulting in an immediately perceivable sensory cue. A method for coating a solid oral dosage form with the pullulan coating composition.

### FIELD OF THE INVENTION

The present invention concerns orally dissolvable film coatings for solid dosage forms. More particularly, the invention relates to a solid dosage form having a core and a coating wherein the coating contains pullulan, one or more sensory cue agents, and a plasticizer.

### BACKGROUND OF THE INVENTION

Pullulan (4,4,6-trigluco-polysaccharide) is a polysaccharide produced from a cultivated fungus of Aureobasidium pullulans, i.e., an α-glucan consisting mainly of maltotriose as repeating units linearly joined through α-1,6-glycosidic linkages. It is usually obtained in the form of an amorphous white powder and is non-toxic, odorless, edible but non-digestible. It is usually used in the fields of foodstuffs and adhesives, and is used in a commercially available product for killing plaque-producing germs as described in U.S. Pat. No. 6,596,298. Pullulan is rarely used in pharmaceutical compositions.

Solid dosage forms have been coated for a variety of reasons including masking objectionable flavors or odors, protecting unstable core compositions, providing protection of the core through the stomach with enteric coatings, improving the appearance of the core, or separating ingredients into a core segment and a coating segment.

Numerous methods for coating solid dosage forms have been developed including sugar-coating, delayed release coating, granule coating, film coating. Often organic solvents are required to prepare these coatings. One popular coating is hydroxypropylmethylcellulose commercially available under the name Opadry.™ One problem with these cellulose film coats such as Opadry™ is that they do not hold volatile ingredients or flavors well.

Even with the foregoing and other core coating compositions, there is still a need for an improved core coating which would provide better mouthfeel, while possessing the ability to hold large amounts of sensory cue agents such as flavor ingredients, particularly volatile flavor ingredients. There is a need for a high volatile ingredient content film coating, that is moist enough so that it is not brittle, but is not so moist that it feels slimy or significantly adheres to adjacent dosage forms.

### SUMMARY OF THE INVENTION

An embodiment of the present invention provides a coating for a solid oral dosage wherein the coating is a pullulan film containing a plasticizer and one or more sensory cue agents.

In another embodiment the invention provides a pullulan film-coated solid oral dosage.

In another embodiment, the invention provides a method for preparing an orally dissolvable film coating, which includes the steps of blending pullulan with plasticizer for a time sufficient to form a homogeneous mixture, and, adding a sensory cue to the homogeneous mixture.

In another embodiment the invention provides a method for making the film-coated solid dosage form by applying the orally dissolvable film in an adherent fashion to a solid core.

In various embodiments of the present invention the film coating is applied over a placebo or at least one systemically acting therapeutic agent in a therapeutically effective amount.

Still further embodiments of the invention provides a method of treating a patient in need of treatment which comprises administering to the patient a therapeutically effective amount of a pullulan film-coated oral dosage form, the dosage form containing a therapeutically effective amount of a drug beneficial to said patient.

Other embodiments of the present invention also provide compositions and methods for administering compositions including combinations of two or more therapeutic agents that promote high patient acceptance and compliance.

### DETAILED DESCRIPTION OF THE INVENTION

Unless otherwise indicated ingredient terms are to be understood to include one or more of the described ingredient.

The present invention is directed to methods and compositions for coating solid dosage forms where the coating is a film containing pullulan, a plasticizer, and a sensory cue agent, wherein the film dissolves in the mouth imparting an immediate sensory effect in the mouth and nasal passages.

Wherein the dosage form contains a therapeutic ingredient, the sensory cue can be formulated such that the patient can perceive the therapeutic ingredient as acting immediately. When emulsified with saliva in the mouth, the film coating releases the sensory cue agents providing stimulating vapors into the nasal passages. Thus, the patient is provided with the sensation of relief although the systemically acting therapeutic agent has not yet begun to alleviate conditions causing the distress. The sensory cue of the coating lasts on the tongue for at least five seconds, which is more than enough time for the tablet to be swallowed before any bitterness in the tablet becomes objectionable.

The coating further provides a slippery texture and a sensation of melting in the mouth which together with the pleasant taste sensations provide for improved patient compliance. The film coating compositions of the present invention are particularly beneficial for patients who have difficulty swallowing, edentulous patients, the elderly, pediatric, bedridden patients, and patients in whom fluid intake must be limited.

“Sensory cue” is defined as a perceptible sensation in the oral/nasal passages. It includes sensations such as prickling, biting, burning, cooling, numbing, heating, vapor action and the like, to a point wherein the patient does not find such sensations objectionable. Sensory cue agents of the present invention include any components which can provide a sensation including pleasant, bitter, tart and the like taste sensations which may or may not be volatile in nature.

Volatile sensory cue agent is defined as any compound having the property of stimulating the thermoreceptors of the nervous system to produce cold or heat sensations. If desired for mouth and throat effects, the sensory cue agent should be volatile. The orally dissolvable film may include nonvolatile sensory cue agents and volatile sensory cue agents as desired.

Sensory cue agents can be selected from substances known to the skilled artisan. Agents know as cooling agents include but are not limited to, menthyl succinate (PHYSCOOL), substituted-p-menthane-3-carboxamides, such as N-ethyl-p-menthane-3-carboximide (WS-3), N,2,3-trimethyl-2-isopropyl butamide (WS-23), 3-1-menthoxy propan-1,2-diol, menthoxypropane diol, menthone glycerol ketal (Frescolat), p-menthane-3,8-diols, cubebol, N,N-dimethyl menthyl succinamide, incilin, menthol, isopulegol, xylitol and others compounds known for their cooling effects and mixtures thereof. Agents which can provide a sensation of heat include but are not limited to capsicum, capsaicinoids, pipperine, gingerols, isothiocyanates, and materials such as chili pepper, horseradish, ginger, black pepper and the like. Sensory cue agents further include essential oils such as, peppermint, wintergreen, eucalyptus, spearmint, cinnamon, clove, bay, thyme, bitter almond, sage, nutmeg, citrus (eg., lemon, orange, lime) and flavoring agents such as eucalyptol, thymol, camphor, methyl salicylate, benzaldehyde, ginger and the like. Sensory cue agents further include acidulants such as citric acid, malic acid and the like. An embodiment uses a cooling agent. The use of menthol is a preferred embodiment.

The amounts of the sensory cue agents added can be readily determined by those skilled in the art. The total amount of sensory cue agents may be from about 0.01 wt. % to about 25 wt. % of the film composition. Generally, the amount of agent will be from about 0.01 to about 4 wt. % of the film composition, preferably about 0.50 to about 3.0 wt. % of the film. The total content should not create sticking or other processing problems.

The film coating can optionally contain one or more flavors such as those described in U.S. Pat. No. 6,596,298 which is incorporated herein. Any amount of flavor can be used generally between about 0.01% to about 10% w/w of the film coating, preferably from 0.01% to 4%. The flavor can be chosen to enhance the sensory cue to add a flavor effect. Likewise the sensory cue agent may be chosen for the flavor effect it can provide.

Pullulan has usually various molecular weights in the range of about 1×10⁴ to 2×10⁶ depending on the processes for the production thereof. The pullulan suitable for the present invention has a molecular weight of about 5×10⁴ to about 1×10⁶, preferably about 7×10⁴ to about 5×10⁵, more preferably about 1×10⁵ to about 3×10⁵. The pullulan can be incorporated into the coating in a wide range of concentrations depending on the molecular weight thereof, but a suitable concentration of pullulan is in the range of about 0.1 to about 20 w/w %, preferably about 2 to about 17 w/w %, particularly preferably about 4 to about 15 w/w %. When the concentration of pullulan is too high, the composition shows unfavorably less surface smoothness.

An advantage of the use of pullulan is superior retention of the volatile sensory cue agents. It would be expected that the high temperatures employed during manufacturing would cause the volatile sensory cue agents to volatilize during the application processes, e.g. spraying or pan coating. The surprising and unexpected result in the actual practice of this invention is that when the volatile sensory cue agents are incorporated into the pullulan coating dispersion, they are retained. In fact, they continue to be retained strong for an unexpectedly long period.

Other water soluble polymers can optionally be added to the coating. Useful water soluble polymers are described in U.S. Pat. No. 6,596,298 to Leung et al. The optional polymer should be chosen so as to not affect the ability to hold volatile sensory cue agents nor affect the flexibility and non-stick properties of the coating.

The coatings of the present invention have high plasticity and favorable mechanical properties. The far better elasticity of pullulan compared with coatings containing cellulose derivatives such as hydroxypropylmethylcellulose (HPMC) or other previously known coatings is advantageous on severe mechanical stress of the coated tablet. The coating composition shows no tackiness either during the application process or during further processing. The coating material is ideally suitable for tableting without damage to the coating. Pullulan shows excellent spreading characteristics and adheres very well to the core tablet.

Further, although HPMC is insoluble in water, it can easily swell and allow water to permeate, resulting in a slimy mouth feel. The coating of the present invention, on the other hand, melts or dissolves resulting in a pleasing or smooth mouthfeel. The great flexibility of the coating means there is no formation of fissures, either during the spraying process or during the tablet swallowing process, through which active ingredient diffuses during passage through the mouth and gives rise to a bitter taste.

The inventors have discovered how to provide a high volatile ingredient content film coating that is moist enough so that it is not brittle, but is not so moist that it feels slimy or significantly adheres to adjacent dosage forms.

One or more plasticizers is added to the film coating to adjust the flexibility and increase the elasticity of the coating to provide a smooth coating that will not crack. The plasticizer provides an improved appearance by eliminating or minimizing cracking, peeling, nicking, picking, off-color and the like. Plasticizers include but are not limited to polyhydric alcohols such as glycerol, sorbitol, mannitol, propylene glycol; esters of polyhydric alcohols such as glycerol triacetate, triacetin, glycerol tricaprylate, monacetin and diacetin and mixtures thereof and the like. The plasticizer of the present invention is preferably an ester of a polyhydric alcohol. Glycerol esters are a particular embodiment. Preferred plasticizers include triacetin.

The plasticizer is added in an amount effective to provide the desired elasticity. Generally, about 0.01 wt/wt % to about 10 wt/wt % plasticizer based on the total pullulan composition used, preferably about 0.1 wt/wt % to about 5 wt/wt %.

One or more surfactants are optionally added to the film coating. Surfactants aid in the dispersion of any oil-based components. Examples of suitable surfactants include, but are not limited to, polysorbates, polyoxyethylene (POE) sorbitan esters, such as POE monooleate (polysorbate 80, Tween 80); sorbitan esters, sorbitan fatty acid esters such as sorbitan monooleate, monolaurate, monostearate, monooleate, and monopalmitate, sorbitan tristearate. It is preferred that the surfactant be selected such that the HLB of the surfactant is in the medium range of from 5-11, preferably from 8-10. A preferred embodiment uses a blend of surfactant. An example of a suitable surfactant would be a combination of a polysorbate and a sorbitan ester. Preferred would be an approximate 1/1 mixture wherein the HLB would be 9-10.

The surfactant is added in an amount effective to provide the desired dispersion. Generally, about 0.1% wt/wt % to about 2.0% wt/wt % surfactant based on the total pullulan composition is used, preferably about 0.25% wt/wt % to about 1.0% wt/wt.

The orally dissolvable film coating may include other pharmaceutically acceptable excipients known to those in the art such as fillers or carriers (e.g. lactose, sucrose, amylose, dextrose, mannitol, inositol), permeabilizing agents, disintegrants, glidants, lubricants, colorants or coloring agents, pH adjusting agents (e.g. fumaric acid, citric acid, sodium acetate), binders (e.g. polyethylene glycols, soluble hydroxyalkylcelluloses, polyvinylpyrrolidone, gelatins, natural gums), and the like. Desirably, the agents are chemically and physically compatible with the active and sensory cue agents.

By “pharmaceutically acceptable” such as the recitation of a “pharmaceutically acceptable excipient,” or a “pharmaceutically acceptable additive”, is meant a material that is not biologically or otherwise undesirable, i.e., the material can be incorporated into a pharmaceutical composition administered to a patient without causing any undesirable biological effects or interacting in a deleterious manner with any of the other components of the composition in which it is contained.

Suitable colorants or coloring agents can be added to the film coatings of the present invention. Illustrative colors or colorants useful herein include without any limitation, pigments, dyes, lakes and oxides and the like. Wherein the dosage form contains a therapeutic agent, the color may be selected to provide a visual signal to complement the sensory cue agent.

The orally dissolvable film coatings of the present invention can include a sweetener. Useful sweeteners include, but are not limited to, sugars such as sucrose, glucose (corn syrup), dextrose, invert sugar, fructose, and mixtures thereof; acid saccharin and its various salts such as the sodium or calcium salt; cyclamic acid and its various salts such as the sodium salt; the dipeptide sweeteners such as aspartame and alitame; sucralose, natural sweeteners such as dihydrochalcone compounds; glycyrrhizin; Stevia rebaudiana (Stevioside); sugar alcohols such as sorbitol, sorbitol syrup, mannitol, xylitol and the like, synthetic sweeteners such as acesulfame-K and sodium and calcium salts thereof and the like, hydrogenated starch hydrolysate (lycasin); protein based sweetening agents such as talin (thaumaoccous danielli) and/or any other pharmacologically acceptable sweetener known by the state of the art, and mixtures thereof.

In a particular embodiment, the formulations according of the invention are free of sugar. A sugar-free formulation has the advantage that it can be administered easily to consumers with blood sugar disorders or to diabetics in need of such preparations. Preferred sweeteners include sucralose, acesulfame potassium, and aspartame which share properties such as absence of bitter and metallic aftertastes.

The total amount of sweetener in a dosage form is between about 0.002 wt/wt % to about 10 wt/wt % of the orally dissolvable film coating. However, this amount can vary greatly depending upon the nature of the composition being sweetened.

A sugar alcohol can be added to further enhance the effect of the volatile sensory cue agent. Suitable sugar alcohols include but are not limited to sorbitol, xylitol, mannitol, galactitol, maltitol, isomalt (PALATINIT™) and mixtures thereof. Typical sugar alcohols are xylitol, mannitol and sorbitol. The exact amount of sugar alcohol employed is a matter of preference subject to such factors as the degree of cooling effect desired. Thus, the amount of sugar alcohol may be varied in order to obtain the result desired in the final product and such variations are within the capabilities of those skilled in the art without the need for undue experimentation.

An effective amount of any generally accepted pharmaceutical lubricant can be added to the film coating. An amount within the range from about 0.25% to about 6%, preferably 0.5% to about 3% by weight can be added. Lubricants are selected from but not limited to the group consisting of highly dispersed silicas, fine particle starches or celluloses and fine particle salts of phosphoric acid or combinations thereof. Examples of suitable lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid. A preferred lubricant is talc.

The orally dissolvable film coating of the present invention can further contain a therapeutically effective amount of therapeutic ingredients. Such therapeutic agents include, but are not limited to, therapeutically effective amounts of, nourishing and health-promoting agents, antipyretic-analgesic-inflammatory agents (such as aspirin, NSAIDS and acetaminophen), antipsychotic drugs, antianxiety drugs, antidepressants, hypnotic-sedative agents, spasmolytics, gastrointestinal function conditioning agents, antacids, antitussive-expectorants (such as dextromethorphan and guaifenesin), dental buccal drugs, antihistamines, smoking sensation agents (such as nicotine), cardiotonics, antiarrhythmic drugs, diuretics, antihypertensive drugs, vasoconstrictors, coronary vasodilators, peripheral vasodilators, cholagogues, antibiotics, chemotherapeutic drugs, antidiabetic agents, drugs for osteoporosis, skeletal muscle relaxants decongestants, demulcent, local anesthetics and mixtures thereof.

The core may be any solid oral dosage form known in the art for the delivery of actives. These forms are those which have a solid carrier such as a polysaccharide matrix or an inorganic material, for example, a dicalcium phosphate and the like, and include but are not limited to tablets, chewable tablets, granules, caplets, wafers, powders, and the like, known in the confectionery and pharmaceutical arts. These forms are distinguished from soft dosage forms such as gelatin capsules, hard gums and the like.

The core may be a placebo or may contain a therapeutic, or active, ingredient as previously described. The active ingredient may be the same as or different from an active ingredient incorporated into the coating.

The present invention may be especially useful in the treatment of respiratory conditions such as upper respiratory indications such as infections, coughs, and asthma. This is because of the sensory cue agents impart an immediate perceivable sensory cue or vaporization effect in the mouth and nasal passages. However, it is emphasized that the invention is not limited to any particular active ingredient or therapeutic category.

In an embodiment, the systemically active therapeutic agent is a nasal decongestant. Examples of suitable systemic nasal decongestants include, but are not limited to, pseudoephedrine, phenylephrine, ephedrine, and phenylpropanolamine.

In order to form the coating on the oral solid dosage form, an aqueous composition is prepared containing at least the pullulan, a plasticizer, and effective amounts of a sensory cue agent. Optionally, the composition may further comprise a surfactant, sweetener, a disintegrating aid, coloring agents, and flavors. The ingredients may be combined in any suitable order, and two or more ingredients may be premixed prior to combining with the remaining ingredients.

The process for forming a film coated core comprises the steps of admixing pullulan, plasticizer, water, and optionally sweeteners and coloring agents under effective shear until dissolved, typically 4-5 hours. The homogeneous mixture should be allowed to stand overnight. In a separate container the aromatic volatile ingredients are mixed with additional processing aids and mixed well. This mixture is added to the pullulan suspension and mixed until uniform. The resulting coating composition is then applied to the core and if necessary, dried.

The core can be panned or spray coated to provide a uniform surface or finish. Other conventional methods of forming the core are within the scope of the invention. For example, an acceptable coating application system includes without limitation, a plain fluid bed system, including a fluid bed spray tower. Also acceptable to prepare the coated core of this invention are a variety of side vented coating pans, spray dryers, continuous coating pans, and conventional coating pans. Any application system capable of applying a composition of this invention to a core is an acceptable system for coating cores employing the pullulan coating composition of this invention.

An effective depth of the film coating is provided for retention. It is also desired that the film coating herein be somewhat resilient with respect to handling, peeling and flaking and being rubbed off the coated tablet.

The pullulan coating composition may be coated onto cores which are uncoated or have been coated with one or more prior coatings (overcoating) of an acceptable coating composition which allows adherency with pullulan. For example, an initial coating may comprise one or more polymers such as celluloses, dextrins, acrylics, colors or other pharmaceutical coating material.

The pullulan coating may be applied as at least one of a primary coating, a secondary coating, or a tertiary coating as desired. One or more coating applications may be made to a coated or uncoated core in accordance with the invention.

The amount of coating provided to the surface of the core is an effective amount and is typically that amount which provides a minimum effective coverage of the exterior surface of the core. Typically the amount of pullulan which is film coated onto cores in practicing this invention is that amount which provides a pullulan film coated core having a weight gain (during coating) from about 1 to 15 w/w % of the total solid oral dosage form and preferably from about 4 to about 10 w/w % of the total solid oral dosage form.

The resulting solid oral dosage form is hard enough to survive in conventional packaging systems such as bottles or blister packs and yet the film coating is dispersible within about 30 seconds in saliva in the mouth.

While certain preferred and alternative embodiments of the invention have been set forth for purposes of disclosing the invention, modification to the disclosed embodiments can occur to those who are skilled in the art.

The following examples serve to provide further appreciation of the invention but are not meant in any way to restrict the effective scope of the invention. All percentages throughout the specification are by weight percent of the final delivery system, unless otherwise indicated.

### EXAMPLES

Pullulan film coating formulations of the invention are set forth below in Tables 1 and 2

TABLE 1

| No. | Component | % w/w Grams |
|---|---|---|
| 1 | Purified water | 32.78 |
| 2 | Green colorant | 0.003 |
| 3 | Acesulfame K | 0.45 |
| 4 | Sucralose | 0.42 |
| 5 | Pullulan | 12.00 |
| 6 | Mint flavor | 2.70 |
| 7 | Physcool | 0.09 |
| 8 | Thymol | 0.13 |
| 9 | Methyl salicylate | 0.19 |
| 10 | Eucalyptol | 0.19 |
| 11 | I-Menthol | 2.20 |
| 12 | Polysorbate 80 | 0.35 |
| 13 | Sorbitan monooleate | 0.35 |
| 14 | Talc | 0.60 |
| 15 | Poly Glycol 600 | 0.72 |
| 16 | Purified water | 46.83 |
| | Total | 100.003 |

Component 1 was weighed into a tared 5 liter container. Components 2, 3, 4, 5 and 15 were added while mixing rapidly. The mixture was mixed approximately 5 hours until all components were dissolved. The solution was allowed to stand overnight. In a separate container components 6, 7, 8, 11, 12 and 13 were mixed until dissolved and uniform. Components 9 and 10 were added with mixing. The second solution was combined with the first and mixed well. Component 14 was added and mixed until uniform. The remaining water 16 was added and mixed for 10 minutes. The viscosity of the final mix was 495 cps on a spindle #2 @ 30 rpm.

TABLE 2

| No. | Component | % w/w Grams |
|---|---|---|
| 1 | Purified water | 33.58 |
| 2 | Acesulfame K | 0.45 |
| 3 | Sucralose | 0.42 |
| 4 | Pullulan | 10.00 |
| 5 | Mint flavor | 2.70 |
| 6 | Physcool | 0.09 |
| 7 | Raspberry flavor | 1.00 |
| 8 | I-Menthol | 2.20 |
| 9 | Polysorbate 80 | 0.25 |
| 10 | Sorbitan monooleate | 0.25 |
| 11 | Talc | 0.60 |
| 12 | Triacetin | 0.50 |
| 13 | Purified water | 47.96 |
| | Total | 100.00 |

Component 1 was weighed into a tared 5 liter container. Components 2, 3, 4 and 12 were added while mixing rapidly. The mixture was mixed approximately 5 hours until all components were dissolved. The solution was allowed to stand overnight. In a separate container components 5, 6, 7, 8, 9 and 10 were mixed until dissolved and uniform. The second solution was combined with the first and mixed well. Component 11 were added and mixed until uniform. The remaining water 13 was added and mixed for 10 minutes. The viscosity of the final mix was 196 cps on a spindle #2 @ 30 rpm.

1. A film-coated solid oral dosage form comprising
a) a solid core
b) an orally dissolvable film coating composition comprising:
i) pullulan;
ii) at least one sensory cue agent; and
iii) a plasticizer;
wherein said film coating is applied to said core.

2. The orally dissolvable film coating composition of claim 1 where said pullulan comprises from about 0.1 to about 20 w/w % of the composition.

3. The orally dissolvable film coating composition of claim 1 wherein said plasticizer is selected from the group consisting of polyhydric alcohols, esters of polyhydric alcohols and mixtures thereof.

4. The orally dissolvable film coating composition of claim 1 wherein said plasticizer is an ester of a polyhydric alcohol.

5. The orally dissolvable film coating composition of claim 1 wherein the plasticizer comprises from about 0.01 wt/wt % to about 10 wt/wt %.

6. The orally dissolvable film coating according to claim 1 wherein at least one sensory cue agent is in amount from about 0.01 to about 25 wt/wt % of the film.

7. The orally dissolvable film coating composition according to claim 1 wherein the sensory cue agent is a volatile sensory cue agent.

8. The orally dissolvable film coating composition according to claim 1 wherein the sensory cue agent is selected from the group consisting of cooling agents, heat sensation agents, essential oils, flavoring agents, acidulants and mixtures thereof.

9. The orally dissolvable film coating composition of claim 1 further comprising a surfactant.

10. The composition according to claim 1 wherein the solid core is selected from the group consisting of tablet, caplet, wafer, granule, powder, and chewable tablet.

11. The composition according to claim 1 where the orally dissolvable film coating comprises from about 1 to 15 w/w % of the total solid oral dosage form.

12. A means for treating a patient comprising administering a pharmaceutical dosage form coated with an orally dissolvable film coating containing pullulan, a plasticizer, and at least one sensory cue agent, such that said sensory cue agent imparts an immediate perceivable sensory cue in the mouth or nasal passages.

13. A method for preparing a film-coated solid oral dosage form comprising the steps of:
a) blending pullulan with a plasticizer for a time sufficient to form a homogeneous mixture;
b) adding volatile sensory cue agents to the homogeneous mixture in a); and
c) applying the mixture to a solid oral dosage form core.
